(19) United States
(12) Patent Application Publication
(10) Pub. No.: US 2010/0003322 A1
(43) Pub. Date: Jan. 7, 2010

(54) ENTERIC COATED HYDROPHOBIC MATRIX FORMULATION

(76) Inventors: FELIX S. LAI, Hayward, CA (US); Richard Ting, San Ramon, CA (US); Christopher A. Simpson, San Jose, CA (US); Bernard Lee, Hayward, CA (US); Sibel Ucpinar, Mountain View, CA (US)

Correspondence Address: HEDMAN & COSTIGAN P.C., 1185 AVENUE OF THE AMERICAS, NEW YORK, NY, 10036, US

(21) Appl. No.: 12/167,876

(22) Filed: Jul. 3, 2008

(51) Int. Cl. A61K 9/28; A61K 31/135; A61K 31/19; A61K 31/195; A61K 31/40; A61K 31/485; A61K 31/34

(52) U.S. Cl. 424/474; 514/646; 514/557; 514/561; 514/423; 514/282; 514/468

(57) ABSTRACT

An enteric coated hydrophobic matrix tablet for soluble, freely soluble and very soluble drugs.

### TECHNICAL FIELD

The present invention relates to a stable controlled release formulation for soluble, freely soluble and very soluble drugs. The formulation employs a core comprising the drug and a hydrophobic rate controlling material. The core is surrounded by an enteric coating. Drugs that can be used in the present invention exhibit a water solubility wherein 35 ml of water or less is required to dissolve 1 gram of drug. Preferred classes of drugs useful in the present invention are anticonvulsants or antiepileptic and opioids.

The dosage form of the present invention is preferably designed for oral administration once or twice a day and to provide therapeutic levels of the soluble drug for about 8 to about 24 hours following administration and preferably about 12 to about 24 hours after administration.

### BACKGROUND OF THE INVENTION

As used in the present application, the term “soluble” refers to compounds that require 35 parts of solvent or less to dissolve 1 part solute and unless otherwise stated encompasses freely soluble and very soluble.

The term “freely soluble” refers to compounds that require 10 parts of solvent or less to dissolve 1 part of solute and the term “very soluble” refers to compounds that require 1 part of solvent or less to dissolve 1 part of solute. Unless otherwise stated herein, the solvent is understood to be water.

Examples of soluble drugs can be found in virtually every therapeutic class such as antidiabetic agents, antihistamine, decongestants, anticonvulsants, cardiovascular agents, stimulants and antibiotics. Some examples of some soluble drugs that are known in the art include but are not limited to buspirone hydrochloride, cefmetazole sodium, cefoxitin sodium, cephapirin sodium, chlorpromazine hydrochloride, cocanine hydrochloride, chlorpheniramine maleate, cyclobenzaprine hydrochloride, diltiazem hydrochloride, diphenhydramine hydrochloride, dopamine hydrochloride, doxylamine succinate, ephedrine hydrochloride, ephedrine sulfate, ephedrine bitartrate, gabapentin, hydroxyamphetamine hydrobromide, levetiracetam, lidocaine hydrochloride, methamphetamine hydrochloride, methylphenidate hydrochloride, metoprolol succinate, metoprolol tartrate, oxybutynin chloride, oxycodone hydrochloride, oxytetracycline hydrochloride, phenylpropanolamine hydrochloride, pilocarpine hydrochloride, pilocarpine nitrate, potassium chloride, pramoxine hydrochloride, pravastatin sodium, prilocain hydrochloride, procaine hydrochloride, ranitidine hydrochloride, pseudoephedrine hydrochloride, sodium valproate and streptomycin sulfate. A more complete index of soluble drugs can be found in Part 7 of Remington: The Science and Practice of Pharmacy, 20^{th} edition, entitled “Pharmaceutical and Medicinal Agents”, and the United States Pharmacopeia 29, which are incorporated herein by reference.

Controlled release dosage forms for soluble drugs are described in the art. For example, diltiazem hydrochloride dosage forms are described in U.S. Pat. Nos. 4,984,240; 5,529,791 and 5,286,497. Controlled release methylphenidate dosage forms are described in U.S. Pat. No. 6,919,373. Similarly, controlled release dosage forms for amphetamine salts are described in U.S. Pat. No. 6,913,768, and pseudoephedrine salts are described in U.S. Pat. No. 5,314,697.

A common controlled release dosage form for soluble drugs employ multiparticulate systems wherein a plurality of small drug cores are prepared and coated with a rate controlling polymeric membrane. Examples of the multiparticulate systems are described in the aforementioned diltiazem hydrochloride patents. The multiparticulate systems are very complicated to make and involve many processing steps.

Another common controlled release dosage form for soluble drugs employs a matrix of hydrophilic material that gels and swells when placed in an aqueous environment. An example of these hydrophilic matrix systems are described in U.S. Pat. Nos. 4,389,393 and 6,340,475. Depending upon the composition, the hydrophilic matrix could rapidly expand upon contact with gastric fluid and thereby remain in the stomach. The retention in the stomach can be undesirable because the drug may degrade in the acidic environment of the stomach or not absorb fully through the stomach lining.

A number of opioids are also soluble. Some examples of soluble opioids include, but are not limited to, codeine phosphate, codeine sulfate, hydromorphone hydrochloride, morphine sulfate, oxycodone hydrochloride, oxymorphone hydrochloride, propoxycaine hydrochloride, propoxyphene hydrochloride and tramadol hydrochloride.

Tramadol is also known as (35 )-trans-2-[dimethylamino)methyl]-1-(3-methoxyphenyl) cyclohexanol) and RR,SS-2-[dimethylamino)methyl]-1-(3-methoxyphenyl) cyclohexanol. Tramadol was first described in U.S. Pat. No. 3,652,589 and is an orally active opioid analgesic. Tramadol is believed to produce an analgesic effect through a mechanism that is neither fully opioid-like nor non-opioid-like because clinical data suggests that tramadol lacks many of the typical side effects of opioid antagonists such as respiratory depression, constipation, tolerance and abuse liability.

U.S. Pat. No. 3,652,589 describes the preparation of acid addition salts of tramadol and in particular the hydrochloride salt. The hydrochloride salt is reported to have a water solubility of about 1 g in 33 ml of water and a duration of action of about 3 to 6 hours following the oral administration of an immediate release composition. A more detailed discussion of the pharmacokinetic and pharmacodynamic properties of tramadol is provided by Lee et al., “Tramadol A Preliminary Review of its Pharmacodynamic and Pharmacokinetics Properties, and Therapeutic Potential in Acute and Chronic Pain States”, Drugs 46 (2), pages 313-340 (1993), the contents of which are incorporated herein by reference.

Immediate release formulations of tramadol have been described in the art. One immediate release oral formulation has been available in the United States for a number of years under the tradename ULTRAM®. Recently, a controlled release form of tramadol has been available in the United States under the tradename ULTRAM® ER. The United States Food and Drug Administration “Approved Drugs and Therapeutic Equivalents” publication, commonly known as the Orange Book, identifies U.S. Pat. No. 6,254,887 as relating to the ULTRAM® ER product.

A number of controlled release opioid products, including tramadol hydrochloride, are also described in the prior art. For example, European Patent Application No. 0 147 780 describes a controlled release formulation that employs polyvinyl alcohol as a controlled release excipient. U.S. Pat. No. 5,968,551 describes controlled release pellet formulations, and U.S. Pat. No. 5,601,842 discloses a sustained release cellulose based matrix tablet. Other oral controlled release opioid dosage forms are disclosed in U.S. Pat. Nos. 4,834,984; 4,990,341; 4,861,598; 5,849,240; 5,965,163; 6,399,096; 6,143,353 and 6,645,527 and United States Published Patent Application Nos. 2007/0003618, 2007/0122478 and 2007/0184115.

A number of anticonvulsants and antiepileptics are also soluble. Some examples of soluble anticonvulsants and antiepileptics include, but are not limited to, gabapentin, levetiracetam and sodium valproate.

Gabapentin is also known as 1-(aminomethyl)cyclohexaneacetic acid. Gabapentin is commercially available under the tradename NEUROTIN® in capsule, tablet and oral solution dosage forms. The package insert for the NEUTROTIN® product describes gabapentin as freely soluble in water and both basic and acidic aqueous solutions.

Levetiracetam is also known as (−)-(S)-α-ethyl-2-oxo-1-pyrrolidine acetamide. Method for preparing levetiracetam are described in U.S. Pat. No. 4,943,639. Levetiracetam is commercially available under the tradename KEPPRA® in tablet and oral solution dosage forms. The package insert for the KEPPRA® product describes levetiracetam as a very soluble compound with approximately 104 grams of the drug being dissolved in 100 ml of water.

Sodium valproate is also known as sodium 2-propylpentanoate, sodium 2-propylvalerate, sodium dipropylacetate and sodium di-n-propylacetate. The chemical and physical properties of sodium valproate are described on pages 529-556 of Analytical Profiles of Drug Substances, Vol. 8, 1979, edited by Klaus Florey. The solubility of sodium valproate is reported on page 543 of the aforementioned reference as one gram in 0.4 ml of water. Sodium valproate is commercially available as an injectable dosage form under the tradename DEPACON®. Controlled release oral dosage forms for sodium valproate are described in U.S. Pat. No. 4,913,906.

Although many attempts have been made to develop safe and effective once-a-day formulations for soluble drugs, many of these attempts have resulted in costly dosage forms that are difficult to manufacture.

It is an object of the present invention to provide a once-a-day tablet dosage form that will release soluble drugs following oral administration over a 12 to 24 hour period.

It is a further object of the present invention to provide controlled release oral tablet dosage forms for soluble drugs that are easy to manufacture.

It is still a further object of the present invention to provide controlled release oral tablet dosage forms for soluble drugs that employ a matrix core free of swelling or hydrogel polymeric material.

It is an additional object of the present invention to provide controlled release tablet dosage forms for soluble drugs that employ a pH dependent coating.

It is an additional objective of the present invention to provide controlled release oral tablet dosage forms for soluble drugs that exhibit a reduced food effect.

It is another object of the present invention to provide controlled release oral tablet dosage forms for freely soluble, preferably very soluble drugs, which employ a matrix core free of swelling or hydrogel polymeric material and very low amounts of diluents or fillers.

These and other objects of the present invention will become apparent from a review of the appended specification.

### SUMMARY OF THE INVENTION

The present invention accomplishes the above objects and others by providing a novel tablet dosage form comprising a therapeutically effective amount of a soluble drug in a controlled release matrix drug core. The matrix drug core is surrounded by an enteric coating.

The matrix drug core preferably comprises a homogeneous mixture of the soluble drug and a hydrophobic rate controlling material that preferably is a solid wax or oil material at room temperature. In a preferred embodiment, the matrix drug core is free of any cellulose material and/or any release controlling excipient that swells or gels upon contact with water.

An alternative embodiment of the present invention comprises, freely soluble, preferably very soluble drugs, wherein the matrix core is free of any cellulose material and/or any release controlling excipient that swells or gels upon contact with water and contains very low amounts of diluents or fillers, i.e less than 10 weight percent and preferably less than 5 weight percent.

The enteric coating surrounding the matrix drug core comprises an enteric or pH dependent material and at least one additional pharmaceutical excipient. As used herein, the term “enteric” and the term “pH dependent material” are used interchangeably to refer to a material that is less soluble in an aqueous media with a lower pH and more soluble in an aqueous media with a higher pH. In a preferred embodiment, the enteric or pH dependent material dissolves or rapidly disperses at a pH level above 5.0, preferably above 5.5 and most preferably above 6.0.

In one embodiment of the present invention, at least one pharmaceutical excipient present in the enteric coating can be any type of coating aid commonly known in the industry such as a plasticizer such as triacetin or acetyltributyl citrate, a film forming polymer such as ethylcellulose, an anti adherent such as talc, a pore forming agent such as a poloxamer, an antifoaming agent, a surfactant, a coloring agent or mixtures of the foregoing. Many of these conventional coating excipients are described in detail in the Handbook of Pharmaceutical Excipients, 4^{th} edition. In one embodiment of the present invention, the at least one pharmaceutical excipient present in the enteric coating is a water soluble material that will dissolve in an aqueous environment regardless of the pH. By adjusting the amount of the enteric coating and the ratio of pH dependent material to water soluble excipient in the enteric coating, the time to maximum plasma concentration (Tmax) of soluble drug following oral administration can be controlled. For example, by decreasing the amount of coating and increasing the ratio of water soluble excipient to pH dependent material, a quicker Tmax can be obtained. Conversely, increasing the amount of enteric coating and decreasing the ratio of water soluble excipient to pH dependent material in the enteric coating will delay the Tmax.

Dosage forms in accordance with the present invention are prepared by conventional tableting and coating procedures. More importantly, the present invention does not require subjecting the intermediates, such as the matrix core, or final enteric coated dosage form to any curing procedures.

As stated previously, one of the objectives of the present invention is to provide a once-a-day tablet that does not exhibit a substantial food effect. Food effect is a phenomenon known in the art where the presence or absence of food will change the bioavailability of a drug. The FDA has recognized that the presence or absence of food with the administration of a drug product can change the bioavailability of the drug and can influence the bioequivalence between a test and a reference product. For example, the FDA has acknowledged that the presence of food during the oral administration of a drug product can affect the bioavailability of the drug by delaying gastric emptying, stimulating bile flow, changing gastrointestinal pH, increasing splanchic blood flow, changing luminal metabolism of a drug substance and/or physically or chemically interacting with a dosage form or a drug substance.

Food effect studies are generally conducted and analyzed according to the conditions and criteria outlined by the FDA in its Guidance for Industry entitled “Food-Effect Bioavailability and Fed Bioequivalence Studies” December of 2002, which is incorporated by reference.

It has been discovered that the use of the enteric coating in the present invention reduces the food effect or variance in bioavailability of the soluble drug when the present invention is administered with or without food according to the test conditions and criteria outlined in the above mentioned FDA Guidance.

### BRIEF DESCRIPTION OF THE DRAWINGS

FIG. 1 is a graph of the in vitro dissolution profiles for the enteric coated tablet described in Example 1 tested with a United States Pharmacopoeia (USP) Type 1 apparatus (basket), at 75 rpms in 900 ml of 0.1 N HCl, 900 ml of deionized water, 900 ml of pH 4.5 phosphate buffer and 900 ml of 6.8 phosphate buffer.

FIG. 2 is a graph of the in vitro dissolution profile of a commercially available lot of ULTRAM® ER (lot P07B037) tested with a USP Type 1 apparatus (basket) at 75 rpms in 900 ml of 0.1 N HCl, 900 ml of deionized water, 900 ml of pH 4.5 phosphate buffer and 900 ml of 6.8 phosphate buffer.

FIG. 3 is the mean plasma concentration profile for Examples 1, 2 and ULTRAM® ER generated from a single dose study conducted under fasting conditions.

FIG. 4 is the mean plasma concentration profile for Examples 1, 2 and ULTRAM® ER generated from a single dose study conducted under fed conditions.

### DETAILED DESCRIPTION OF THE INVENTION

The subject invention concerns a stable enteric coated pharmaceutical tablet formulation comprising a therapeutically effective amount of a soluble drug. Examples of soluble drugs that can be used in the present invention include but are not limited to buspirone hydrochloride, cefmetazole sodium, cefoxitin sodium, cephapirin sodium, chlorpromazine hydrochloride, cocanine hydrochloride, chlorpheniramine maleate, cyclobenzaprine hydrochloride, diltiazem hydrochloride, diphenhydramine hydrochloride, dopamine hydrochloride, doxylamine succinate, ephedrine hydrochloride, ephedrine sulfate, ephedrine bitartrate, gabapentin, hydroxyamphetamine hydrobromide, levetiracetam, lidocaine hydrochloride, methamphetamine hydrochloride, methylphenidate hydrochloride, metoprolol succinate, metoprolol tartrate, oxybutynin chloride, oxycodone hydrochloride, oxytetracycline hydrochloride, phenylpropanolamine hydrochloride, pilocarpine hydrochloride, pilocarpine nitrate, potassium chloride, pramoxine hydrochloride, pravastatin sodium, prilocain hydrochloride, procaine hydrochloride, ranitidine hydrochloride, pseudoephedrine hydrochloride, sodium valproate, streptomycin sulfate. A more complete list of soluble drugs can be found in Part 7 of Remington: The Science and Practice of Pharmacy, 20^{th} edition, entitled, “Pharmaceutical and Medicinal Agents”, and the United States Pharmacopeia 29, both of which are incorporated herein by reference.

One class of drugs useful in the present invention is anticonvulsants or antiepileptics. Examples of some anticonvulsants or antiepileptics include but are not limited to gabapentin, levetiracetam and sodium valproate.

Another class of drugs useful in the present invention is the class of drugs commonly known as opioids. Examples of some of the opioids useful in the present invention include, but are not limited to, codeine phosphate, codeine sulfate, hydromorphone hydrochloride, morphine sulfate, oxycodone hydrochloride, oxymorphone hydrochloride, propoxycaine hydrochloride, propoxyphene hydrochloride and tramadol hydrochloride.

One of the more preferred drugs useful in the present invention are the pharmaceutically acceptable salts of tramadol. The preferred pharmaceutically acceptable salt is the hydrochloride salt. Unless otherwise indicated, the term “tramadol” as used throughout this specification includes racemic mixtures, as well as the individual isomers and soluble pharmaceutically acceptable salts thereof. The amount of tramadol in the enteric coated tablet of the present invention can range from 25 mg to 500 mg. The preferred amounts are 100 mg, 200 mg or 300 mg.

Another of the preferred drugs useful in the present invention is levetiracetam. Unless otherwise indicated, the term “levetiracetam” as used throughout this specification includes racemic mixtures, as well as the individual isomers and soluble pharmaceutically acceptable salts thereof.

The enteric coated tablets of the present invention comprise a matrix tablet or tablet core coated with an enteric coating. The matrix tablet or core comprises: (i) the soluble, freely soluble or very soluble drug; (ii) a hydrophobic rate controlling excipient; and (iii) a diluent. The matrix tablet or core is prepared by mixing the aforementioned ingredients and compressing the mixture into a tablet.

Another embodiment of the present invention includes enteric coated tablets comprising a matrix tablet or tablet core coated with an enteric coating. The matrix tablet or core comprises: (i) a freely soluble or very soluble drug (ii) a hydrophobic rate controlling excipient; and (iii) optionally a diluent. The matrix tablet or core is prepared by mixing the aforementioned ingredients and compressing the mixture into a tablet. In this embodiment, the diluent is optional and if present should only be present in an amount of 0 to about 10 weight percent, preferably 0 to about 5 weight percent.

The hydrophobic rate controlling excipient employed in the matrix tablet or core of the present invention is preferably a wax or oil material that is a solid at room temperature. The preferred wax or oil material used in the present invention is also a non-polymeric material and is water insoluble. In one embodiment of the invention, the wax or oil material should also have a melting point greater than 50° C., preferably between about 55° C. and about 150° C., most preferably between about 70° C. and about 100° C.

Examples of the rate controlling wax or oil material that are useful in the present invention include beeswax, white wax, emulsifying wax, hydrogenated vegetable oil, hydrogenated castor oil, microcrystalline wax, cetyl alcohol, stearyl alcohol, free wax acids such as stearic acid, esters of wax acids, propylene glycol monostearate, glycerol monostearate, carnauba wax, palm wax, candelilla wax, lignite wax, ozokerite, ceresin wax, lardaceine and China wax. Other possible rate controlling excipients useful in the present invention include saturated hydrocarbons having 25 to 31 carbon atoms, saturated alcohols having from 25 to 31 carbon atoms, saturated monocarboxylic acids having from 25 to 31 carbon atoms, esters obtained from said alcohols and monocarboxylic acids which are described in U.S. Pat. No. 6,923,984 and incorporated herein by reference.

Diluents that may be used in the matrix tablet or core of the present invention include any material that does not delay the release of the soluble drug from the matrix core. The diluent is preferably a water soluble material that will dissolve when the matrix tablet or core is placed in an aqueous environment. The dissolution of the diluent should create pores or channels in the matrix tablet or core. Some examples of diluents that may be used in the present invention include sugars such as sucrose, lactose, mannitol and sorbitol, organic acids such as fumaric or citric acid, low viscosity water soluble polymers, (i.e., less than 15 mPa s, preferably less than 10 mPa s for a 2% solution at 20° C.) such as hydroxypropyl methylcellulose, polyvinyl alcohol or polyvinylpyrrolidone, and salts such as sodium chloride or potassium chloride.

In one embodiment of the present invention, the diluent is a water soluble polymer with a melting point below 100° C., preferably about 40° C. to about 75° C. An example of such a water soluble polymeric diluent with a low melting point is polyethylene glycol. Polyethylene glycols that are particularly useful in the present invention have an average molecular weight between 500 and 8000, preferably between 1000 and 5000 and most preferably between 2000 and 4000.

The matrix tablet or core of the present invention may also optionally include anti-adherents, lubricants, glidants and other common pharmaceutical excipients. Examples of some anti-adherents, lubricants and glidants that may be used in the present invention include talc, magnesium stearate, calcium stearate, stearic acid, hydrogenated vegetable oils, polyethylene glycols, silicon dioxide and mixtures of the foregoing. In a preferred embodiment of the present invention, the lubricant has a melting point of about 40° C. to about 100° C., preferably about 45° C. to about 85° C.

The matrix tablet or core of the present invention is prepared by mixing the drug, the hydrophobic rate controlling material, optionally the diluent and optionally the lubricant and/or glidant by any conventional methods known in the tableting art. For example, a blender or high shear mixer can be used to mix the matrix tablet or core components prior to tableting. The matrix tablet or core components may also be granulated by wet or dry granulation techniques as well as extrusion and spheronization prior to tableting.

In one embodiment of the present invention, the matrix tablet or core is prepared by melting the hydrophobic rate controlling material and adding the soluble drug and diluent to the melt. Alternatively, the soluble drug, hydrophobic rate controlling material and diluent are mixed in a blender or high speed mixer then heated until the rate controlling material is melted. In both of these embodiments, the soluble drug becomes uniformly dispersed or suspended in the melted material. The melted liquid with the uniformly dispersed drug is subsequently cooled to a solid and milled to create drug granules.

In an alternate embodiment, the soluble drug granules can be prepared by spray drying the melt suspension. An example of a spray drying technique is described in PCT Patent Application No. WO 93/17667 entitled “Composition for Oral Preparations”, which is incorporated herein by reference.

If a lubricant is employed in the matrix tablet or core, it may be mixed with the core materials prior to the melting of the hydrophobic rate controlling material or after the hydrophobic rate controlling material has been melted but prior to cooling. In one embodiment of the present invention, the lubricant is selected so it also melts with the rate controlling material during preparation of the soluble drug granules.

Once the drug granules are prepared, they are optionally mixed with a glidant such as silicon dioxide and compressed into the matrix tablet or core.

The compressed controlled release matrix tablets or cores are subsequently coated with an enteric coating. The enteric coating comprises an enteric or pH dependent material and at least one pharmaceutical excipient, preferably a water soluble excipient.

The enteric or pH dependent materials useful in the present invention do not dissolve until they encounter an aqueous media with a pH of about 5.0 or higher, preferably about 5.5 or higher, and most preferably about 6.0 and higher. Representative examples of the enteric or pH dependent material that can be used in the present invention include cellulose acetate phthalate, hydroxypropylmethyl cellulose phthalate, hydroxypropyl methylcellulose acetate succinate, polyvinyl acetate phthalate, cellulose acetate trimelitate, carboxymethylethylcellulose, methyacrylic acid copolymers such as, Eudragit L (polymethacrylic acid, methylmethacrylate), 1:1 ratio, MW (No. Av. 135,000—USP Type A) or Eudragit S (polymethacrylic acid, methylmethacrylate), 1:2 ratio, MW (No. Av. 135,000—USP Type B), shellac, zein and mixtures thereof. The preferred enteric material is a methacrylic acid copolymer Type B, commercially available as EUDRAGIT® S 100.

The enteric coating should also comprise at least one pharmaceutical excipient other than the enteric or pH dependent material. The one additional pharmaceutical excipient can be selected from plasticizers, film forming polymers, anti adherents, lubricants, pore forming agents, coloring agents, antifoaming agents, surfactants or mixtures of the foregoing.

Suitable anti-adherents and lubricants are described above. Suitable plasticizers include acetyl triethyl citrate, dibutyl phthalate, tributyl citrate, triethyl citrate, acetyl tributyl citrate, propylene glycol, triacetin, polyethylene glycol and diethyl phthalate.

Suitable pore forming agents include sodium chloride, potassium chloride, sucrose, sorbitol, mannitol, polyethylene glycols (PEG), propylene glycol, hydroxypropyl cellulose, hydroxypropyl methycellulose, polyvinyl alcohols, methacrylic acid copolymers, poloxamers (such as LUTROL F68, LUTROL F127 and LUTROL F108 which are commercially available from BASF) and mixtures thereof.

The enteric coating can be applied to the matrix tablet or core by any method commonly used in the art such as pan coating. It is important that the coating temperature and conditions be kept below the melting temperature of the release controlling material of the core.

In one embodiment of the present invention, the enteric coating will comprise the enteric or pH dependent material and a water soluble pharmaceutical excipient in an amount that will create pores or channels in the enteric coating after administration to a mammal, preferably a human. The water soluble excipient should be present in an amount sufficient to allow the enteric coated tablet to exhibit the following dissolution profile when tested in 900 ml 0.1 N HCl using a USP Type 2 apparatus (paddle) at 100 rpms:

| Time (hours) | Preferred | Most Preferred |
|---|---|---|
| 2 | 1-20% | 2-15% |
| 4 | 5-45% | 10-35% |
| 6 | 10-60% | 15-50% |
| 8 | 20-75% | 25-65% |
| 12 | 25-90% | 30-80% |

In an alternate embodiment of the present invention employing a freely soluble or very soluble drug in the tablet core, the final enteric coated tablet may exhibit the following dissolution profile when tested in 900 ml 0.1 N HCl using a USP Type 2 apparatus (paddle) at 100 rpms:

| Time (hours) | Preferred | Most Preferred |
|---|---|---|
| 2 | 5-50% | 10-45% |
| 4 | 15-75% | 20-70% |
| 8 | 20-95% | 25-90% |
| 12 | NLT 90% | NLT 95% |

NLT = not less than

The final dosage form of the present invention may also include an immediate release amount of drug if desired. The immediate release component can be in the form of an immediate release layer applied to the enteric coating.

The enteric coated tablet of the present invention may also optionally include a final aesthetic color and/or polishing coat. The aesthetic or polishing coat can be applied to the enteric coated tablet by any method commonly used in the art such as pan coating. It is important that the coating temperature and conditions be kept below the melting temperature of the release controlling material of the core.

An embodiment of the present invention should have the following matrix tablet or core composition:

| Matrix Tablet or Core: | Preferred | Most Preferred |
|---|---|---|
| Soluble Drug | 5-70% | 10-60% |
| Rate Controlling Agent | 5-60% | 10-50% |
| Diluent | 5-60% | 10-50% |
| Lubricant | 0-10% | 0.5-5% |
| Glidant | 0-10% | 0.5-5% |

The above percentages are based upon the total weight of the matrix tablet or core.

An alternate embodiment of the present invention for freely soluble, preferably very soluble drugs should have the following matrix tablet or core composition:

| Matrix Tablet or Core: | Preferred | Most Preferred |
|---|---|---|
| Freely/Very Soluble Drug | 15-75% | 20-70% |
| Rate Controlling Agent | 5-60% | 10-50% |
| Diluent | 0-10% | 0-5% |
| Lubricant | 0-10% | 0.5-5% |
| Glidant | 0-10% | 0.5-5% |

The above percentages are based upon the total weight of the matrix tablet or core.

An embodiment of the present invention should have the following enteric coating composition:

| Enteric Coating: | Preferred | Most Preferred |
|---|---|---|
| pH dependent material | 25-90% | 35-75% |
| pore forming agent | 0-20% | 0-15% |
| plasticizer | 0-25% | 0.5-15% |
| anti-adherent | 0-50% | 0.5-45% |

The above percentages are based upon the total weight of the enteric coating.

The final enteric coated tablet of an embodiment of the present invention that comprises a soluble, a freely soluble or a very soluble drug should exhibit the following in vitro dissolution profile when tested in a USP Type 1 apparatus (basket) at 75 rpms in 900 ml of 0.1 N HCl at 37° C.:

| Time (hours) | Preferred | Most Preferred |
|---|---|---|
| 2 | 0-25% | 2-20% |
| 4 | 5-50% | 10-40% |
| 8 | 20-70% | 25-65% |
| 16 | 35-95% | 40-90% |
| 24 | NLT 70% | NLT 80% |

NLT = NOT LESS THAN

The final enteric coated tablet of an embodiment of the present invention that comprises a soluble, a freely soluble or a very soluble drug should also exhibit the following in vitro dissolution profile when tested in a USP Type 1 apparatus (basket) at 75 rpms in 900 ml of deionized water at 37° C.:

| Time (hours) | Preferred | Most Preferred |
|---|---|---|
| 2 | 0-20% | 2-15% |
| 4 | 3-45% | 7-40% |
| 8 | 15-60% | 20-50% |
| 16 | 25-80% | 30-75% |
| 24 | NLT 65% | NLT 70% |

NLT = NOT LESS THAN

The final enteric coated tablet of an embodiment of the present invention that comprises a soluble, a freely soluble or a very soluble drug should further exhibit the following in vitro dissolution profile when tested in a USP Type 1 apparatus (basket) at 75 rpms in 900 ml of 4.5 phosphate buffer at 37° C.:

| Time (hours) | Preferred | Most Preferred |
|---|---|---|
| 2 | 0-20% | 2-15% |
| 4 | 3-45% | 7-40% |
| 8 | 15-60% | 20-50% |
| 16 | 25-80% | 30-75% |
| 24 | NLT 60% | NLT 65% |

NLT = NOT LESS THAN

The final enteric coated tablet of an embodiment of the present invention that comprises a soluble, a freely soluble or a very soluble drug should exhibit the following in vitro dissolution profile when tested in a USP Type 1 apparatus (basket) at 75 rpms in 900 ml of 6.8 phosphate buffer at 37° C.:

| Time (hours) | Preferred | Most Preferred |
|---|---|---|
| 2 | 0-25% | 2-20% |
| 4 | 5-50% | 10-40% |
| 8 | 20-70% | 25-65% |
| 16 | 35-95% | 40-90% |
| 24 | NLT 70% | NLT 80% |

NLT = NOT LESS THAN

In an alternate embodiment of the present invention, wherein the drug in the tablet core is a freely soluble or very soluble drug, the final enteric coated tablet may exhibit the following in vitro dissolution profile when tested in a USP Type 1 apparatus (basket) at 75 rpms in 900 ml of 0.1 N HCl at 37° C.:

| Time (hours) | Preferred | Most Preferred |
|---|---|---|
| 2 | 10-70% | 15-60% |
| 4 | 20-85% | 30-80% |
| 8 | 40-95% | 50-95% |
| 12 | NLT 90% | NLT 95% |

NLT = NOT LESS THAN

In an alternate embodiment of the present invention, wherein the drug in the tablet core is a freely soluble or very soluble drug, the final enteric coated tablet may also exhibit the following in vitro dissolution profile when tested in a USP Type 1 apparatus (basket) at 75 rpms in 900 ml of 6.8 phosphate buffer at 37° C.:

| Time (hours) | Preferred | Most Preferred |
|---|---|---|
| 2 | 10-70% | 15-60% |
| 4 | 20-85% | 30-80% |
| 8 | 40-95% | 50-95% |
| 12 | NLT 90% | NLT 95% |

NLT = NOT LESS THAN

### Description of the Preferred Embodiments

The following are provided by way of example only and are by no means intended to be limiting.

### EXAMPLE 1

A 100 mg enteric coated tramadol HCl tablet in accordance with the present invention was prepared as follows:

### Matrix Tablet or Core

Approximately 9.6 kg of carnauba wax is heated to approximately 85° C. using a water-jacked melter. Once the carnauba wax is melted, 12.0 kg of Tramadol HCl, 12.0 kg of polyethylene glycol 3350 and 0.4 kg of stearic acid are added to the melt and mixed for approximately 15 minutes. The mixture is cooled and milled with a Fitzmill equipped with a 0.065″ screen to create approximately 34.0 kg of tramadol granules. Approximately 5 kg of the tramadol granules are blended with 0.7 kg of silicon dioxide for approximately 1 minute then milled with a Fitzmill equipped with a 0.065″ screen. The tramadol granules that have been mixed with the silicon dioxide and milled are blended with the remaining 29 kg of tramadol granules in a 5 cubic foot slant cone blender for approximately 13 minutes. The blended material is compressed into 0.3150″ round tablets using a rotary tablet press.

### Enteric Coating

An enteric coating suspension was prepared by mixing approximately 0.6 kg of EUDRAGIT® S100, 0.06 kg of triethyl citrate, 0.3 kg of talc, 8.3 kg of isopropyl alcohol and 0.4 kg of water. The enteric coating suspension was applied to approximately 34.7 kg of the matrix tablets or cores prepared above using a perforated pan coater. After the enteric coating suspension was applied, the coated tablets were dried in the pan coater for about 20 minutes with an inlet air temperature of about 45° C.

### Cosmetic Coating

A cosmetic coating suspension comprising approximately 1.1 kg of OPRADY II, White and 5.6 kg of water was prepared. The cosmetic coating suspension was applied to approximately 35.6 kg of the enteric coated tablets prepared above using a perforated pan coater. After the cosmetic coating suspension was applied, the coated tablets were dried in the pan coater for about 30 minutes with an inlet air temperature of about 45° C.

The final dosage form had the following composition:

| Ingredient | %(w/w) | mg/tablet |
|---|---|---|
| Tramadol HCl | 32.67 | 100.0 |
| Carnauba Wax, NF | 26.13 | 80.0 |
| Polyethylene Glycol, NF | 32.67 | 100.0 |
| Stearic Acid, NF | 1.09 | 3.33 |
| Silicon Dioxide, NF | 1.89 | 5.78 |
| EUDRAGIT ® S100 | 1.65 | 5.06 |
| Triethyl Citrate | 0.17 | 0.51 |
| Talc, USP | 0.83 | 2.53 |
| OPADRY II, White | 2.91 | 8.92 |

The enteric coated tablet prepared in Example 1 was tested using a USP Type 1 apparatus (basket) at 75 rpms, 37° C. in 900 ml of 0.1 N HCl, deionized water, pH 4.5 phosphate buffer and pH 6.8 phosphate buffer. The results of this testing are as follows:

| Time (hours) | 0.1 N HCl | D.I. Water | pH 4.5 | pH 6.8 |
|---|---|---|---|---|
| 2 | 8.7% | 5.8% | 5.2% | 7.4% |
| 4 | 21.7% | 16.3% | 15.3% | 19.3% |
| 8 | 41.3% | 34.7% | 32.0% | 38.7% |
| 10 | 49.4% | 42.5% | 38.7% | 47.0% |
| 16 | 67.5% | 61.2% | 56.8% | 67.0% |
| 24 | 83.1% | 78.1% | 73.7% | 83.2% |
| 36 | 94.3% | 92.3% | 89.8% | 93.0% |

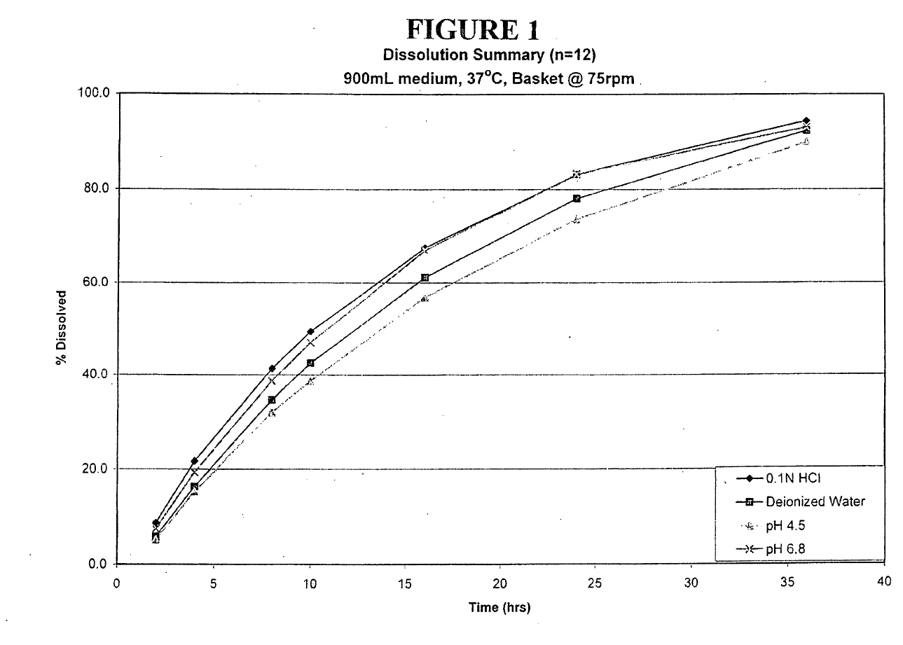

The results of the dissolution testing on the enteric coated tablet of Example 1 are graphically shown in FIG. 1.

### EXAMPLE 2

A 100 mg enteric coated tablet in accordance with the present invention was prepared according to the procedure described in Example 1. The final 100 mg tablet had the following composition:

| Ingredient | % (w/w) | mg/tablet |
|---|---|---|
| Tramadol HCl | 32.67 | 100.0 |
| Carnauba Wax, NF | 27.77 | 85.0 |
| Polyethylene Glycol, NF | 31.03 | 95.0 |
| Stearic Acid, NF | 1.09 | 3.33 |
| Silicon Dioxide, NF | 1.89 | 5.78 |
| EUDRAGIT ® S100 | 1.65 | 5.06 |
| Triethyl Citrate | 0.17 | 0.51 |
| Talc, USP | 0.83 | 2.53 |
| OPADRY II, White | 2.91 | 8.92 |

The enteric coated tablets of Example 1, Example 2 and ULTRAM® ER, a commercially available extended release tramadol tablet, were tested in vivo according to standard FDA bioequivalency testing procedures. A general description of the in vivo testing procedures can be found in the FDA documents entitled “Guidance for Industry-Bioavailability and Bioequivalence Studies for Orally Administered Drug Products-General Considerations” March 2003 and/or “Guidance for Industry-Food-Effect Bioavailability and Fed Bioequivalence Studies” December 2002, which are incorporated herein by reference. The data from the in vivo studies was analyzed using standard statistical procedures such as outlined in the FDA documents entitled “Statistical Procedures for Bioequivalence Studies Using a Standard Two-Treatment Crossover Design” July 1992 and/or “Statistical Approaches to Establishing Bioequivalence”, which are incorporated herein by reference.

The results of a single dose cross over study conducted on Examples 1, 2 and ULTRAM® ER are as follows:

MEAN PHARMACOKINETIC PARAMETERS (FASTED) (n = 24)

| Parameter | Example 1 | Example 2 | ULTRAM ® ER |
|---|---|---|---|
| Tₘₐₓ (hr) | 9.25 | 8.917 | 9.875 |
| Cₘₐₓ (ng/ml) | 147.790 | 135.093 | 146.010 |
| AUCₗₐₛₜ (ng * hr/ml) | 2679.302 | 2565.380 | 2686.294 |
| AUC_{0-∞} (ng * hr/ml) | 2747.828 | 2679.473 | 2762.840 |

MEAN PHARMACOKINETIC PARAMETERS (FED) (n = 24)

| Parameter | Example 1 | Example 2 | ULTRAM ® ER |
|---|---|---|---|
| Tₘₐₓ (hr) | 13.4585 | 14.792 | 11.917 |
| Cₘₐₓ (ng/ml) | 103.760 | 98.262 | 118.469 |
| AUCₗₐₛₜ (ng * hr/ml) | 2411.582 | 2316.486 | 2441.560 |
| AUC_{0-∞} (ng * hr/ml) | 2514.364 | 2427.627 | 2536.678 |

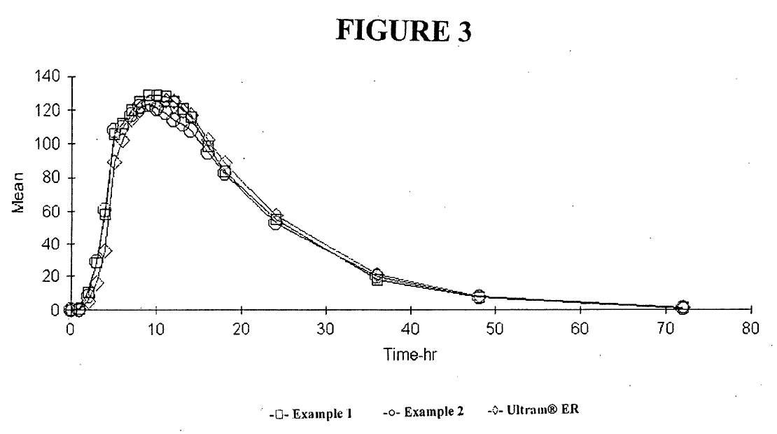

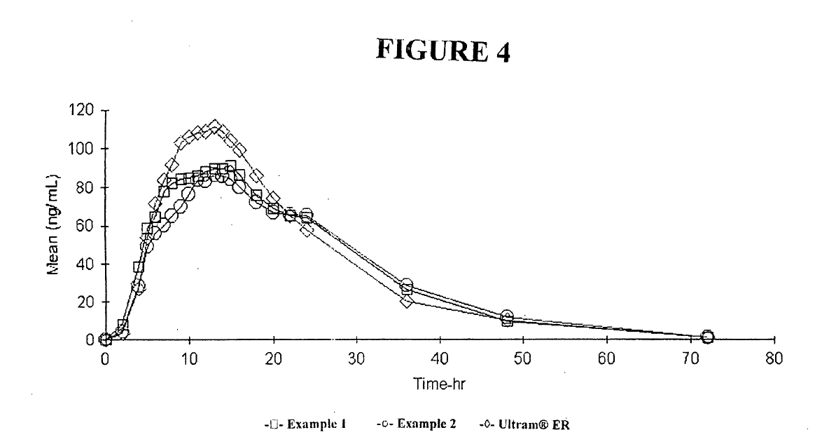

The mean plasma concentration time graphs for the above described in vivo testing are shown in FIGS. 3 and 4.

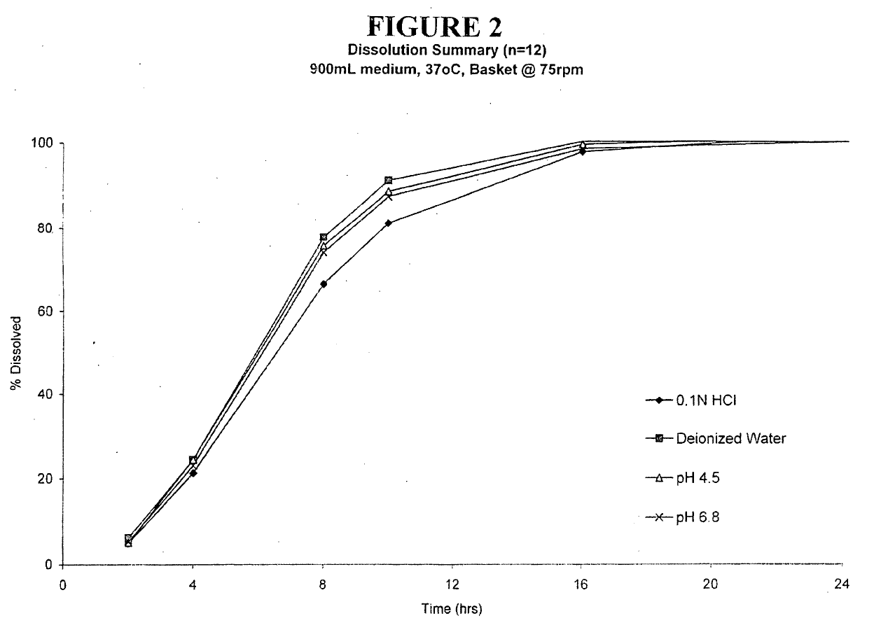

For comparison purposes, the in vitro dissolution profiles for the ULTRAM® ER product employed in the above-described biostudies are shown in FIG. 2. The actual values from the dissolution testing shown in FIG. 2 are as follows:

| Time | | | | |
|---|---|---|---|---|
| (hours) | 0.1N HCl | D.I Water | pH 4.5 | pH 6.8 |
| 2 | 4.9% | 6.2% | 5.1% | 5.3% |
| 4 | 21.3% | 24.3% | 24.4% | 23.0% |
| 8 | 66.5% | 77.8% | 75.8% | 74.2% |
| 10 | 81.3% | 91.5% | 88.8% | 87.7% |
| 16 | 98.3% | 100.7% | 100.0% | 99.0% |
| 24 | 102.3% | 100.7% | 101.8% | 100.8% |
| 36 | 102.7% | 101.0% | 102.7% | 101.0% |

### EXAMPLE 3

A 200 mg enteric coated tablet in accordance with the present invention was prepared according to the procedure described in Example 1. The final 200 mg tablet had the following composition:

| Ingredient | %(w/w) | mg/tablet |
|---|---|---|
| Tramadol HCl | 30.63 | 200.0 |
| Carnauba Wax, NF | 45.95 | 300.0 |
| Polyethylene Glycol, NF | 15.32 | 100.0 |
| Stearic Acid, NF | 1.02 | 6.67 |
| Silicon Dioxide, NF | 1.90 | 12.38 |
| EUDRAGIT ® S100 | 1.42 | 9.29 |
| Triethyl Citrate | 0.14 | 0.93 |
| Talc, USP | 0.71 | 4.64 |
| OPADRY II, White | 2.91 | 19.02 |

The enteric coated tablet prepared in Example 3 was tested using a USP Type 2 apparatus (paddle) at 100 rpms, 37° C. in 900 ml of 0.1 N HCl. The results of this testing are as follows:

| Time (hours) | 0.1 N HCl |
|---|---|
| 2 | 8% |
| 4 | 17% |
| 8 | 34% |
| 10 | 41% |
| 16 | 57% |
| 24 | 72% |
| 30 | 80% |

### EXAMPLE 4 (PROPHETIC)

A 4 mg, 8 mg or 16 mg enteric coated hydromorphone hydrochloride tablet in accordance with the present invention can be prepared according to the procedure described in Example 1. The final tablets may have the following composition:

| Ingredient | % (w/w) |
|---|---|
| Hydromorphone HCl | 20-40 |
| Carnauba Wax, NF | 30-60 |
| Polyethylene Glycol, NF | 5-25 |
| Stearic Acid, NF | 0.5-5 |
| Silicon Dioxide, NF | 0.5-5 |
| EUDRAGIT ® S100 | 0.5-5 |
| Triethyl Citrate | 0.05-3 |
| Talc, USP | 0.05-3 |
| OPADRY II, White (optional) | 0-5 |

### EXAMPLE 5 (PROPHETIC)

An enteric coated oxycodone hydrochloride tablet with 30 mg to 200 mg of oxycodone hydrochloride can be prepared in accordance with the present invention by the procedure described in Example 1. The final tablets may have the following composition:

| Ingredient | % (w/w) |
|---|---|
| Oxycodone HCl | 20-40 |
| Carnauba Wax, NF | 30-60 |
| Polyethylene Glycol, NF | 5-25 |
| Stearic Acid, NF | 0.5-5 |
| Silicon Dioxide, NF | 0.5-5 |
| EUDRAGIT ® S100 | 0.5-5 |
| Triethyl Citrate | 0.05-3 |
| Talc, USP | 0.05-3 |
| OPADRY II, White (optional) | 0-5 |

### EXAMPLE 6 (PROPHETIC)

An enteric coated morphine sulfate tablet with 15 mg to 150 mg of morphine sulfate can be prepared in accordance with the present invention by the procedure described in Example 1. The final tablets may have the following composition:

| Ingredient | % (w/w) |
|---|---|
| Morphine Sulfate | 20-40 |
| Carnauba Wax, NF | 30-60 |
| Polyethylene Glycol, NF | 5-25 |
| Stearic Acid, NF | 0.5-5 |
| Silicon Dioxide, NF | 0.5-5 |
| EUDRAGIT ® S100 | 0.5-5 |
| Triethyl Citrate | 0.05-3 |
| Talc, USP | 0.05-3 |
| OPADRY II, White (optional) | 0-5 |

### EXAMPLE 7 (PROPHETIC)

An enteric coated gabapentin tablet with 100 mg to 1000 mg of gabapentin can be prepared in accordance with the present invention by the procedure described in Example 1. The final tablets may have the following composition:

| Ingredient | % (w/w) |
|---|---|
| Gabapentin | 25-60 |
| Carnauba Wax, NF | 30-60 |
| Polyethylene Glycol, NF | 0-10 |
| Stearic Acid, NF | 0.5-5 |
| Silicon Dioxide, NF | 0.5-5 |
| EUDRAGIT ® S100 | 0.5-5 |
| Triethyl Citrate | 0.05-3 |
| Talc, USP | 0.05-3 |
| OPADRY II, White (optional) | 0-5 |

### EXAMPLE 8 (PROPHETIC)

An enteric coated levetiracetam tablet with 500 mg of levetiracetam can be prepared in accordance with the present invention by the procedure described in Example 1. The final tablets may have the following composition:

| Ingredient | % (w/w) |
|---|---|
| Levetiracetam | 55-70 |
| Carnauba Wax, NF | 15-35 |
| Stearic Acid, NF | 0.5-3 |
| Silicon Dioxide, NF | 0.5-3 |
| Enteric Polymer | 0.5-5 |
| Triethyl Citrate | 0.05-3 |
| Talc, USP | 0.01-3 |
| OPADRY II, White (optional) | 0-5 |

### EXAMPLE 9 (PROPHETIC)

An enteric coated levetiracetam tablet with 100 mg to 1000 mg of levetiracetam can be prepared in accordance with the present invention by the procedure described in Example 1. The final tablets may have the following composition:

| Ingredient | % (w/w) |
|---|---|
| Levetiracetam | 30-70 |
| Hydrophilic Rate Controlling Excipient | 30-60 |
| Diluent | 0-10 |
| Lubricant | 0.5-5 |
| Glidant | 0.5-5 |
| Enteric Polymer | 0.5-5 |
| Plasticizer | 0.05-3 |
| Anti-Adherent | 0.05-3 |
| OPADRY II, White (optional) | 0-5 |

### EXAMPLE 10 (PROPHETIC)

An enteric coated levetiracetam tablet with 500 mg and 1000 mg of levetiracetam can be prepared in accordance with the present invention by the procedure described in Example 1. The final tablets may have the following composition:

| Ingredient | % (w/w) |
|---|---|
| Levetiracetam | 50-75 |
| Hydrophilic Rate Controlling Excipient | 15-40 |
| Diluent | 0-10 |
| Lubricant | 0.5-5 |
| Glidant | 0.5-5 |
| Enteric Polymer | 0.5-5 |
| Plasticizer | 0-5 |
| Anti-Adherent | 0-5 |
| Coloring Agent (optional) | 0-5 |

### EXAMPLE 11 (PROPHETIC)

An enteric coated sodium valproate tablet with 250 mg to 1000 mg of sodium valproate can be prepared in accordance with the present invention by the procedure described in Example 1. The final tablets may have the following composition:

| Ingredient | % (w/w) |
|---|---|
| Sodium Valproate | 25-60 |
| Carnauba Wax, NF | 30-60 |
| Polyethylene Glycol, NF | 0-10 |
| Stearic Acid, NF | 0.5-5 |
| Silicon Dioxide, NF | 0.5-5 |
| EUDRAGIT ® S100 | 0.5-5 |
| Triethyl Citrate | 0.05-3 |
| Talc, USP | 0.05-3 |
| OPADRY II, White (optional) | 0-5 |

The prophetic examples described in Examples 7-11 if tested using a USP Type 2 apparatus (paddle) at 100 rpms, 37° C. in 900 ml of 0.1 N HCl, and pH 6.8 phosphate buffer should exhibit the following dissolution profile:

| Time | | |
|---|---|---|
| (hours) | 0.1 N HCl | pH 6.8 |
| 0.5 | 5-25% | 15-25% |
| 1 | 10-35% | 20-35% |
| 2 | 25-50% | 35-50% |
| 4 | 40-75% | 55-75% |
| 8 | 60-95% | 75-95% |
| 12 | NLT 90% | NLT 90% |

While certain preferred and alternative embodiments of the present invention have been set forth for purposes of disclosing the invention, modifications to the disclosed embodiments may occur to those who are skilled in the art. In addition, based upon the foregoing description and published literature, an individual of ordinary skill will understand that a pharmaceutical excipient can exhibit different properties depending upon the concentration in the dosage form or the manner in which it is formulated. For example, it is reported in the literature that microcrystalline cellulose can act as a tablet disintegrant at concentrations of 5-15% but as a tablet binder or diluent at concentrations of 20-90%. Accordingly, the appended claims are intended to cover all embodiments of the invention and modifications thereof which do not depart from the spirit and scope of the invention.

1. An enteric coated tablet comprising:
(a) a matrix drug core comprising:
(i) a therapeutically effective amount of a soluble drug;
(ii) a hydrophobic rate controlling material that is a solid at room temperature; and
(iii) optionally a diluent and
(b) an enteric coating surrounding the matrix drug core comprising:
(i) a pH dependent material, and
(ii) at least one pharmaceutical excipient.

2. The enteric coated tablet as defined in claim 1 wherein the rate controlling material is a wax or oil and has a melting point greater than 50° C.

3. The enteric coated tablet as defined in claim 2 wherein the rate controlling wax or oil has melting point of about 55° C. to about 150° C.

4. The enteric coated tablet as defined in claim 2 wherein the rate controlling wax or oil has a melting point of about 70° C. to about 100° C.

5. The enteric coated tablet as defined in claim 1 wherein the diluent is water soluble.

6. The enteric coated tablet as defined in claim 1 wherein the diluent is water soluble and has a melting point below 100° C.

7. The enteric coated tablet as defined in claim 6 wherein the diluent has a melting point of about 40° C. to about 75° C.

8. The enteric coated tablet as defined in claim 1 wherein the matrix drug core further comprises a lubricant.

9. The enteric coated tablet as defined in claim 8 wherein the lubricant has a melting point of about 40° C. to about 100° C.

10. The enteric coated tablet as defined in claim 8 wherein the lubricant has a melting point of about 45° C. to about 85° C.

11. The enteric coated tablet as defined in claim 1 wherein the pH dependent material is methacrylic acid copolymer Type B.

12. The enteric coated tablet as defined in claim 1 wherein the pharmaceutical excipient present in the enteric coating is a plasticizer.

13. The enteric coated tablet as defined in claim 1 wherein the pharmaceutical excipient present in the enteric coating is an anti-adherent.

14. The enteric coated tablet as defined in claim 1 wherein the soluble drug is an opioid.

15. The enteric coated tablet as defined in claim 14 wherein the opioid is selected from the group consisting of codeine phosphate, codeine sulfate, hydromorphone hydrochloride, morphine sulfate, oxycodone hydrochloride, oxymorphone hydrochloride, propoxycaine hydrochloride, propoxyphene hydrochloride and tramadol hydrochloride.

16. The enteric coated tablet as defined in claim 1 wherein the soluble drug is a pharmaceutically acceptable salt of tramadol.

17. The enteric coated tablet as defined in claim 16 wherein the drug is tramadol hydrochloride.

18. The enteric coated tablet as defined in claim 1 wherein the drug is freely soluble.

19. The enteric coated tablet as defined in claim 1 wherein the drug is very soluble.

20. The enteric coated tablet as defined in claim 1 wherein the drug is an anticonvulsant or antiepileptic.

21. The enteric coated tablet as defined in claims 20 wherein the anticonvulsant or antiepileptic is selected from the group consisting of gabapentin, levetiracetam, and sodium valproate.

22. An enteric coated tablet consisting essentially of:
(a) a matrix drug core consisting essentially of:
(j) 5-70 weight percent based upon the weight of the matrix core of a soluble drug;
(ii) 5-60 weight percent based upon the weight of the matrix core of a rate controlling, non-polymeric, water insoluble wax with a melting point of about 55° C. to about 150° C.;
(iii) 5-60 weight percent based upon the weight of the matrix core of a water soluble diluent with a melting point of about 40° C. to about 75° C.;
(iv) 0-10 weight percent based upon the weight of the matrix core of a lubricant; and
(v) 0-10 weight percent based upon the weight of the matrix core of a glidant; and
(b) an enteric coating surrounding the matrix drug core comprising:
(i) 25-90 weight percent based upon the weight of the enteric coating of a pH dependent material;
(ii) 0-25 weight percent based upon the weight of the enteric coating of a plasticizer; and
(iii) 0-50 weight percent based upon the weight of the enteric coating of an anti-adherent.

23. The enteric coated tablet as defined in claim 22 wherein the soluble drug is an opioid.

24. The enteric coated tablet as defined in claim 23 wherein the opioid is selected from the group consisting of codeine phosphate, codeine sulfate, hydromorphone hydrochloride, morphine sulfate, oxycodone hydrochloride, oxymorphone hydrochloride, propoxycaine hydrochloride, propoxyphene hydrochloride and tramadol hydrochloride.

25. The enteric coated tablet as defined in claim 22 wherein the soluble drug is a pharmaceutically acceptable salt of tramadol.

26. The enteric coated tablet as defined in claim 25 wherein the drug is tramadol hydrochloride.

27. An enteric coated tablet consisting essentially of:
(a) a matrix drug core consisting essentially of:
(k) 15-75 weight percent based upon the weight of the matrix core of a freely soluble or very soluble drug;
(ii) 5-60 weight percent based upon the weight of the matrix core of a rate controlling, non-polymeric, water insoluble wax with a melting point of about 55° C. to about 150° C.;
(iii) 0-10 weight percent based upon the weight of the matrix core diluent;
(iv) 0-10 weight percent based upon the weight of the matrix core of a lubricant; and
(v) 0-10 weight percent based upon the weight of the matrix core of a glidant; and
(b) an enteric coating surrounding the matrix drug core comprising:
(j) 25-90 weight percent based upon the weight of the enteric coating of a pH dependent material; and
(ii) 0-50 weight percent based upon the weight of the enteric coating of at least one pharmaceutical excipient.

28. The enteric coated tablet as defined in claim 27 wherein the drug is an anticonvulsant, an antiepileptic or an opioid.

29. The enteric coated tablet as defined in claim 28 wherein the drug is an anticonvulsant or antiepileptic selected from the group consisting of gabapentin, levetiracetam and sodium valproate.

30. The enteric coated tablet as defined in claim 27 wherein the diluent comprises 0 to about 5 weight percent based upon the weight of the matrix core.
